(19) United States
(12) Patent Application Publication
(10) Pub. No.: US 2003/0108556 A1
(43) Pub. Date: Jun. 12, 2003

(54) Therapeutic uses of polyvalent compositions in infectious diseases

(76) Inventors: John J. Mekalanos, Charlestown, MA (US); Ying Wang, Brookline, MA (US); R. John Collier, Wellesley Hills, CA (US); Michael Mourez, Boston, MA (US)

Correspondence Address: EDWARDS & ANGELL, LLP, P.O. BOX 9169, BOSTON, MA, 02209, US

(21) Appl. No.: 10/165,762

(22) Filed: Jun. 7, 2002

Related U.S. Application Data
(60) Provisional application No. 60/296,942, filed on Jun. 8, 2001.

(51) Int. Cl. A61K 39/00; A61K 39/38

(52) U.S. Cl. 424/184.1

(57) ABSTRACT

New therapeutic methods and compositions are provided for treating against an infectious agent in a mammal by administration of a polymeric material having linked thereto a plurality of therapeutic agents against the infective agent, wherein the polymer comprises polymerized dextran or ethylene glycol units. The compositions and methods of the invention are particularly useful to treat against bacterial infections, including treatment of mammalian cells infected with gram-negative bacteria or gram-positive bacteria. The compositions of the invention can be useful for treating against anthrax, staphylococcus, pneumococcus and other bacteria, parasites, fungi, viral and protozoan infections.

This application claims benefit of U.S. Provisional patent application, serial No. 60/296,942, filed Jun. 8, 2001, which is incorporated herein by reference.

### FIELD OF THE INVENTION

The invention includes therapeutic methods that comprise administration of specific inhibitors of toxins or other moieties to cells produced by an infectious agent. In particular, the invention provides methods for treatment of infectious diseases and disorders caused by viruses, bacteria, protozoa, fungi. Preferred administered inhibitors are based on multiple copies of peptides or oligonucleotides, specific for toxins or other moieties, being displayed on a polymeric backbone. These polyvalent inhibitors disrupt the binding of infectious disease agent peptides on a site of the cell-binding moiety related to the binding site of the enzymatic moieties. Various toxins including Anthrax toxin, Diphtheria toxin and Pseudomonas exotoxin A are suitable toxins for such therapy.

### BACKGROUND OF THE INVENTION

Anthrax toxin is produced by Bacillus anthracis, the causative agent of anthrax, and is responsible for the major symptoms of the disease¹. Clinical anthrax is rare, but there is growing concern over the potential use of B. anthracis in biological warfare and terrorism. Although a vaccine against anthrax exists, various factors make mass vaccination impractical. The bacteria can be eradicated from the host by treatment with antibiotics, but because of the continuing action of the toxin, such therapy is of little value once symptoms have become evident. Thus, a specific inhibitor of the action of the toxin might prove a valuable adjunct to antibiotic therapy.

Bacillus anthracis produces three proteins which when combined appropriately form two potent toxins, collectively designated anthrax toxin. Protective antigen, a single receptor-binding moiety (PA, 82,684 Da (Dalton)) and edema factor, an enzymatic moiety (EF, 89,840 Da) combine to form edema toxin (ET), while PA and lethal factor (LF, 90,237 Da) another enzymatic moiety, combine to form lethal toxin (LT) (Leppla, S. H. Alouf, J. E. and Freer, J. H., eds. Academic Press, London 277-302, 1991). ET and LT each conform to the AB toxin model, with PA providing the target cell binding (B) function and EF or LF acting as the effector or catalytic (A) moieties. A unique feature of these toxins is that LF and EF have no toxicity in the absence of PA, apparently because they cannot gain access to the cytosol of eukaryotic cells.

After release from the bacteria as nontoxic monomers, these three proteins diffuse to the surface of mammalian cells and assemble into toxic, cell-bound complexes. Cleavage of PA into two fragments by a cell-surface protease enables the fragment that remains bound to the cell, PA63, to heptamerize³ and bind EF and LF with high affinity (Kd˜1 nM). After internalization by receptor-mediated endocytosis, the complexes are trafficked to the endosome. There, at low pH, the PA moiety inserts into the membrane and mediates translocation of EF and LF to the cytosol. EF is an adenylate cyclase that has an inhibitory effect on professional phagocytes, and LF is a protease⁴ that acts specifically on macrophages, causing their death and the death of the host.

The genes for each of the three anthrax toxin components have been cloned and sequenced (Leppla, 1991). This showed that LF and EF have extensive homology in amino acid residues 1-300. Since LF and EF compete for binding to PA63, it is highly likely that these amino-terminal regions are responsible for binding to PA63. Direct evidence for this was provided in a mutagenesis study (Quinn et al. J. Biol. Chem. 266:20124-20130, 1991); all mutations made within amino acid residues 1-210 of LF led to decreased binding to PA63. The same study also suggested that the putative catalytic domain of LF included residues 491-776 (Quinn et al., 1991). In contrast, the location of functional domains within the PA63 polypeptide is not obvious from inspection of the deduced amino acid sequence. However, studies with monoclonal antibodies and protease fragments (Leppla, 1991) and subsequent mutagenesis studies (Singh et al. J. Biol. Chem. 266:15493-15497, 1991) indicated that residues at and near the carboxyl terminus of PA are involved in binding to receptor.

PA is capable of binding to the surface of many types of cells. After PA binds to a specific receptor (Leppla, 1991) on the surface of susceptible cells, it is cleaved at a single site by a cell surface protease, furin, to produce an amino-terminal 19-kDa fragment that is released from the receptor/PA complex (Singh et al. J. Biol. Chem. 264:19103-19107, 1989). Removal of this fragment from PA exposes a high-affinity binding site for LF and EF on the receptor-bound 63-kDa carboxyl-terminal fragment (PA63). Cleavage of PA occurs after residues 164-167, Arg-Lys-Lys-Arg. This site is also susceptible to cleavage by trypsin and can be referred to as the trypsin cleavage site. Only after cleavage is PA able to bind either EF or LF to form either ET or LT. The complex of PA63 with LF and/or EF is endocytosed and is trafficked to acidified endosomes. There the PA63 moiety inserts into the membrane and forms a pore, and the LF and EF moieties cross the membrane to the cytosol, where they modify cytosolic substrates.

Prior work had shown that the carboxyl terminal PA fragment (PA63) can form ion conductive channels in artificial lipid membranes (Blaustein et al. Proc. Natl. Acad. Sci. U.S.A. 86:2209-2213, 1989; Koehler, T. M. and Collier, R. J. Mol. Microbiol. 5:1501-1506, 1991), and that LF bound to PA63 on cell surface receptors can be artificially translocated across the plasma membrane to the cytosol by acidification of the culture medium (Friedlander, A. M. J. Biol. Chem. 261:7123-7126, 1986). Furthermore, drugs that block endosome acidification protect cells from LF (Gordon et al. J. Biol. Chem. 264:14792-14796, 1989; Friedlander, 1986; Gordon et al. Infect. Immun. 56:1066-1069, 1988). The mechanisms by which EF is internalized have been studied in cultured cells by measuring the increases in cAMP concentrations induced by PA and EF (Leppla, S. H. Proc. Natl. Acad. Sci. U.S.A. 79:3162-3166, 1982; Gordon et al., 1989). However, because assays of cAMP are relatively expensive and not highly precise, this is not a convenient method of analysis. Internalization of LF has been analyzed only in mouse and rat macrophages, because these are the only cell types lysed by the lethal toxin.

Another toxin which causes serious side effects is Pseudomonas exotoxin A (PE). The sequence is deposited with GenBank. Structural determination by X-ray diffraction, expression of deleted proteins, and extensive mutagenesis studies have defined three functional domains in PE: a receptor-binding domain (residues 1-252 and 365-399) designated Ia and Ib, a central translocation domain (amino acids 253-364, domain II), and a carboxyl-terminal enzymatic domain (amino acids 400-613, domain III). Domain III catalyzes the ADP-ribosylation of elongation factor 2 (EF-2), which results in inhibition of protein synthesis and cell death. It has also been suggested that an extreme carboxyl terminal sequence is essential for toxicity (Chaudhary et al. Proc. Natl. Acad. Sci. U.S.A. 87:308-312, 1990; Seetharam et al. J. Biol. Chem. 266:17376-17381, 1991). Since this sequence is similar to the sequence that specifies retention of proteins in the endoplasmic reticulum (ER) (Munro, S. and Pelham, H. R. B. Cell 48:899-907, 1987), it was suggested that PE must pass through the ER to gain access to the cytosol. Detailed knowledge of the structure of PE has facilitated use of domains II, lb, and III (together designated PE40) in hybrid toxins and immunotoxins.

Although, antibiotics may eradicate the bacteria, the harmful effects of the infection may not be removed because of the continuing action of the toxin. Thus, such therapy is of little value once symptoms have become evident. In addition, antibiotic resistant strains are continually emerging thereby, exacerbating attempts for treatment. There is thus, a need for alternative forms of therapy or therapy that can be used adjunct to antibiotic therapy, such as specific inhibitors of the action of toxin.

### SUMMARY OF THE INVENTION

We now provide new therapeutic methods and compositions for treating against an infectious agent in a mammal by administration of a polymeric material having linked thereto a plurality of therapeutic agents against the infective agent, wherein the polymer comprises polymerized dextran or ethylene glycol units.

The compositions and methods of the invention are particularly useful to treat against bacterial infections, including treatment of mammalian cells infected with gram-negative bacteria or gram-positive bacteria. The compositions of the invention can be particularly effective for treating against anthrax, staphylococcus, pneumococcus and other bacteria, parasites, fungi, viral and protozoan infections.

According to one preferred embodiment of the invention, the polyvalent molecule inhibits for example, viral replication; a viral infection cycle, such as, for example, attachment to cellular ligands; viral molecules encoding host immune modulating functions. Particularly preferred viral organisms causing human diseases according to the present invention include (but not restricted to) Herpes viruses, Hepatitisviruses, Retroviruses, Orthomyxoviruses, Paramyxoviruses, Togaviruses, Picomaviruses, Papovaviruses and Gastroenteritisviruses.

According to another preferred embodiment of the invention, the polyvalent molecule is specific for human or domestic animal bacterial pathogens. Particularly preferred bacteria causing serious human diseases are the Gram positive organisms: Staphylococcus aureus, Staphylococcus epidermidis, Enterococcus faecalis and E. faecium, Streptococcus pneumoniae and the Gram negative organisms: Pseudomonas aeruginosa, Burkholdia cepacia, Xanthomonas maltophila, Escherichia coli, Enterobacter spp, Klebsiella pneumoniae and Salmonella spp. The polyvalent molecule may target molecules that may include (but are not restricted to) genes or proteins essential to bacterial survival and multiplication in the host organism, virulence genes or proteins, genes encoding single- or multi-drug resistance.

According to one preferred embodiment of the invention, the polyvalent molecule is specific for protozoa infecting humans and causing human diseases. Particularly preferred protozoan organisms causing human diseases according to the present invention include (but not restricted to) Malaria e.g. Plasmodium falciparum and M. ovale, Trypanosomiasis (sleeping sickness) e.g. Trypanosoma cruzei, Leischmaniasis e.g. Leischmania donovani, Amebiasis e.g. Entamoeba histolytica.

According to one preferred embodiment of the invention, the polyvalent molecule is specific for fingi causing pathogenic infections in humans. Particularly preferred fingi causing human diseases according to the present invention include (but not restricted to) Candida albicans, Histoplasma neoformans, Coccidioides immitis and Penicillium marneffei.

Preferred linked therapeutic agents of the compositions and methods of the invention are biologically active peptides, although other pharmaceutically active compounds can be employed including non-peptidic small molecules and polynucleic acid compounds.

Such therapeutic agents can be suitably covalently linked to a polymer systems having dextran or ethylene glycol units. The polymeric scaffolding also may be further functionalized to provide desired physical characteristics, e.g. by linkage of pendant hydrophobic and/or hydrophilic moieties.

In particular, the composition is comprised of a polymer covalently linked to multiple therapeutic agents or ligand. When the target protein is present at a high density on the surface of cell or other biological surface, it is possible to increase the biological activity of a weakly binding ligand by presenting multiple copies of it on the same molecule. Preferred polymeric backbones are flexible so that structural constraints are not an issue as different micro environments in the animal's body have different pH levels, different chemistries, such as hydrophobic, hydrophilic ad the like. Polymers of the compositions and methods of the invention also may be cross-linked to another polymer, thereby further increasing the multivalency of peptide or other therapeutic agent groups.

A particular preferred polymeric backbone is comprised is comprised of dextran polymers of varying lengths. A preferred length is comprised of at least about forty dextran monomers to about two hundred dextran monomers. To increase the valency of a peptide of interest, a plurality of peptide units are covalently linked to the polymeric backbone. A preferred ratio of peptide unit to monomer is at least about one peptide unit per ten monomers to at least about one peptide unit per fifty monomers.

Another preferred polymeric backbone is a poly(ethylene glycol) molecule. The poly(ethylene glycol) backbone is comprised of at least about forty poly(ethylene glycol) molecules to at least about two hundred poly(ethylene glycol) molecules. The poly(ethylene glycol) backbone is preferably covalently linked to multiple peptide units. A preferred ratio of peptide units to poly(ethylene glycol) molecules is at least about one peptide unit per ten poly(ethylene glycol) molecules to at least about one peptide unit per fifty poly(ethylene glycol) molecules.

In one aspect, the polymeric backbones are comprised of pendant moieties which increase the hydrophobicity or hydrophilicity of the polymeric backbone. The polymeric backbones may also be cross-linked to another backbone polymer, thereby increasing the multivalency of peptide units.

The polyvalent therapeutic agents such as peptide, preferably have the ability to interfere with the assembly and/or functionality of a toxin of an infectious disease agent. In particular, in the case of anthrax, preferably the therapeutic agents (e.g. peptides) can inhibit the function of the heptameric complex of anthrax toxin. A mechanism of action would be, for example, where the peptide units interfere with the binding of edema factor and lethal factor of the anthrax toxin, inhibit the function of the heptameric complex of anthrax toxin. Heptatmerization of the anthrax toxin is necessary for the functionality of the toxin.

Particularly preferred compositions where peptides of the same sequence are linked to a polymer. However, multiple peptides of differing sequence also may be suitably linked to a single polymer. Such an approach may be preferred e.g. where more than one type of infectious disease agent produces multiple toxins. Preferably, more than one type of polyvalent toxin inhibitor can be administered during the course of treatment.

In specifically preferred embodiments of the invention, inhibitors of anthrax toxin have been employed and administered to protect cells and animal challenged with this toxin. The inhibitors are based on multiple copies of peptides binding PA being displayed on a polymeric backbone.

Without being bound by any theory, the polyvalent molecules inhibit anthrax toxin association by inhibiting interaction of enzymatic and cell binding moieties of the toxin. This disruption is believed due to the binding of the peptides on a site of the cellbinding moiety related to the binding site of the enzymatic moieties. As detailed in the examples which follow, one tested inhibitor was able to prevent the action of the toxin in an animal model and it is the first synthetic molecule able to do so.

The polyvalent inhibitor comprised of the polymeric backbone and plurality of peptide units is particularly useful in treatment of patients infected with for example, gram positive or gram negative bacteria or bacteria that may be resistant to antibiotics. The present composition can also be administered to a mammal in need of such therapy in conjunction with other therapies such as antibiotics, chemotherapy and the like.

Other aspects of the invention are discussed infra.

### BRIEF DESCRIPTION OF THE DRAWINGS

FIG. 1 is a diagram illustrating the anthrax intoxication process: 1. Binding of PA to its receptor. 2. Proteolytic activation of PA and dissociation of PA20. 3. Self-association of monomeric PA63 to form the heptameric prepore. 4. Binding of EF/LF to the prepore. 5. Endocytosis of the receptor:PA63:ligand complex. 6. pH-dependent insertion of PA63 and translocation of the ligand. The polyvalent inhibitors described in this report blocked step 4.

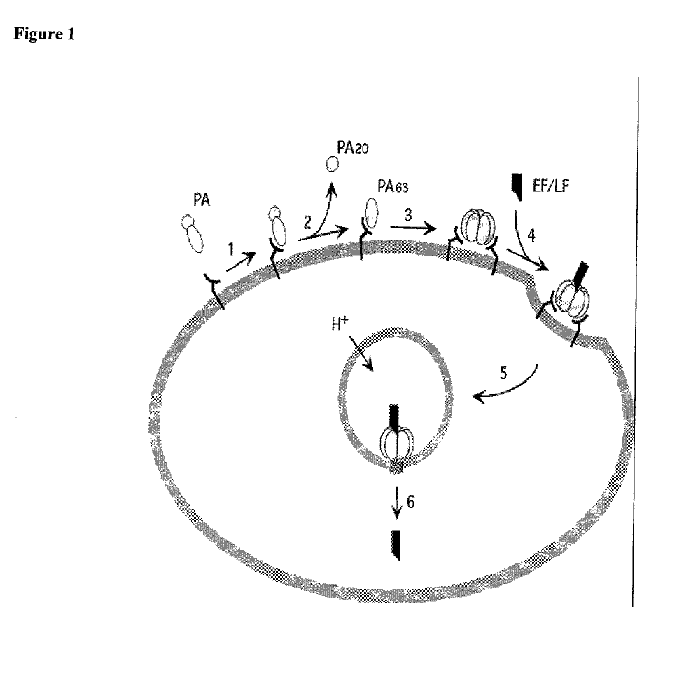

FIG. 2 is an illustrative example of the selection of phage displaying heptamer specific peptides: the phage library binds the PA63 heptamer coated on the plastic surface of a tube (step1). After extensive washes (step2), a first elution is performed with PA83 (step3) in order to remove phages that would bind surfaces that are common to PA83 and PA63. Remaining phages represented the phages binding surfaces that are specific to the heptamer. These phages were recovered by eluting with an excess of soluble PA63 heptamer (step4).

FIG. 3 is a graph depicting the results of an ELISA of selected peptide displaying phages.

FIG. 4 is a graph illustrating the inhibition of LFnDTA and PA toxicity by inhibitors based on dextran backbones.

### DETAILED DESCRIPTION OF THE INVENTION

The present invention provides compositions and methods for treating an infection in a mammal by administering to the mammal a therapeutically effective amount of an inhibitor of the toxin produced by the infecting agent, such as for example, the toxin produced when a mammal is infected by Bacillus anthracis. The inhibitor is comprised of multiple peptides linked to a flexible backbone. The backbone of the polyvalent molecule is comprised of a polymer, where the polymer contains polymerized dextran or ethylene glycol units. Polyvalent presentation of specific toxin inhibitors results in a high efficiency of toxin activity inhibition.

Polymers provide a versatile framework system and are preferably used as a presenter of multiple units of a peptide referred to herein as a polyvalent molecule presenter or polyvalent inhibitor. The two terms are used interchangeably throughout the disclosure.

As used herein, a “polyvalent molecule presenter” refers to a polymer, such as a derivative of dextran or poly(ethylene glycol) that has multiple, covalently linked copies of the peptide of interest, for example, an anthrax toxin inhibitor. At least one peptide unit is preferably covalently linked to a polymer, more preferably about ten peptide units, most preferably at least about 25 peptide units. A polyvalent molecule presenter may present a plurality of the same peptide units or may present a plurality of dissimilar peptide units or heterologous components.

A “heterologous” component refers to a component that is introduced into or produced within a different entity from that in which it is naturally located. For example, a polynucleotide derived from one organism and introduced by genetic engineering techniques into a different organism is a heterologous polynucleotide which, if expressed, can encode a heterologous polypeptide. Similarly, a promoter or enhancer that is removed from its native coding sequence and operably linked to a different coding sequence is a heterologous promoter or enhancer.

As used herein, “plurality of peptide units” is comprised of at least five peptide units of the same sequence.

As used herein, “dissimilar peptide units” are peptides that vary by at least one amino acid.

As used herein, a “peptide unit” refers to a sequence of amino acids comprised of at least two amino acids, more preferably at least about eight amino acids, most preferably at least about twelve amino acids. The sequence of the amino acids is determined by the ability of the peptide unit to inhibit a toxin of interest, for example, peptide units that inhibit anthrax toxin assembly.

The terms “polypeptide,” “peptide,” and “protein” are used interchangeably to refer to polymers of amino acids of any length. These terms also include proteins that are post-translationally modified through reactions that include glycosylation, acetylation and phosphorylation.

The terms “variant” and “amino acid sequence variant” are used interchangeably and designate polypeptides in which one or more amino acids are added and/or substituted and/or deleted and/or inserted at the N- or C-terminus or anywhere within the corresponding native sequence. In various embodiments, a “variant” polypeptide usually has at least about 75% amino acid sequence identity, or at least about 80% amino acid sequence identity, preferably at least about 85% amino acid sequence identity, even more preferably at least about 90% amino acid sequence identity, and most preferably at least about 95% amino acid sequence identity with the amino acid sequence of the corresponding native sequence polypeptide.

Identification of peptide units that have the ability to inhibit toxins are identified by use of phage display techniques. This technique are well known in the art. See, for example, Gordon et al., Nature, 395:710-713, 1998; Smith et al, Science 228:1315 (1985). Phage screening kits are also available commercially. For example, a commercial library of peptides displayed on the surface of a bacteriophage M13 can be purchased from New England Biolabs.

Screening peptide libraries is a proven strategy for identifying inhibitors of protein-ligand interactions. Compounds identified in these screens often bind to their targets with low affinities. When the target protein is present at a high density on the surface of cell or other biological surface, it is sometimes possible to increase the biological activity of a weakly binding ligand by presenting multiple copies of it on the same molecule. An example of isolating a peptide for inhibiting anthrax toxin, as disclosed herein, is not meant to limit the invention in any way but serves merely to illustrate a method for isolating possible inhibitors of toxins or other proteins that are part of the cause of action of an infectious agent in the disease process. In this illustrative example a peptide is isolated from a phage display library that binds weakly to the heptameric cell-binding subunit of anthrax toxin and prevents the interaction between cell-binding and enzymatic moieties. A molecule consisting of multiple copies of this non-natural peptide, covalently linked to a flexible backbone, prevented assembly of the toxin complex in vitro and blocked toxin action in an animal model. This result is the first demonstration of inhibition of protein-protein interactions by a synthetic, polymeric, polyvalent inhibitor in vivo. A detailed description of identification of the peptide units is found in the examples which follow.

As used herein, “therapeutic potency” is a measure of the capacity of the polyvalent molecule presenter to inhibit the formation of toxin complexes, as defined by the in vitro inhibition assay of toxin action in cell cultures, which is described in the materials and methods section.

Specifically preferred aspects of the invention include use of inhibitors of anthrax toxin. Preferred inhibitors are based on multiple copies of peptides binding PA being displayed on a polymeric backbone. The polyvalent molecules prevent anthrax toxin association by preventing interaction of enzymatic and cell binding moieties of the toxin. Without being bound by theory, this disruption is due to the binding of the peptides on a site of the cell-binding moiety related to the binding site of the enzymatic moieties.

The polymers of the present invention can be prepared via, direct polymerization or copolymerization of a monomer, and nucleophilic side chain substitution on a activated polymer. The monomers can be polymerized using, for example, methods of free radical polymerization which are well known in the art. Due to reactivity differences between the two monomers, the mole ratio of the monomers in the copolymer product can be different from the mole ratio of the monomers in the initial reaction mixture. This reactivity difference can also result in a non-random distribution of monomers along the polymer chain.

The polymers of the present invention are comprised of homopolymers or copolymers, and can have, for example, a dextran, poly(ethylene glycol) or polyacrylamide backbone. In one embodiment, the polymers of the present invention include copolymers which comprise a hydrophobic monomer and, optionally, one or more additional monomers, such as neutral hydrophilic monomers. As used herein, the term “polymer backbone” or “backbone” refers to that portion of the polymer which is a continuous chain, comprising the bonds which are formed between monomers upon polymerization. The composition of the polymer backbone can be described in terms of the identity of the monomers from which it is formed, without regard to the composition of branches, or side chains, off of the polymer backbone. Thus, a dextran polymer is said to have a dextran backbone. Preferably, the dextran backbone is comprised of at least about forty monomers, more preferably about 200, most preferably at least about 500 monomers.

The term “monomer”, as used herein, refers to both (a) a single molecule comprising one or more polymerizable functional groups prior to or following polymerization, and (b) a repeat unit of a polymer. An unpolymerized monomer capable of addition polymerization, can, for example, comprise an olefinic bond which is lost upon polymerization. A copolymer is said to comprise two or more different monomers.

The term “pendant”, as used herein, refers to a structural component of one or more polymer side chains or groups which is not a part of the polymer backbone. Therefore, polymers of the present invention comprise side chains or groups. Preferred groups are ethanolamine, tryptophan or benzylamine.

The polymers comprising the backbone of the polyvalent molecule presenter can be cross-linked, for example, by incorporation of a multifunctional co-monomer, thereby increasing the valency of the presenting molecule. Most preferred are about two polymer backbones crosslinked, each presenting at least one peptide. The amount of cross-linking agent is typically between 0.5% and 25% by weight relative to the weight of the polymer, preferably from about 2.5% to about 20% by weight.

Polymers bearing amino groups can be cross-linked by bridging units between amino groups on adjacent polymer strands. Suitable bridging units include straight chain or branched, substituted or unsubstituted alkylene groups, diacylalkylene groups and diacylarene groups. Examples of suitable bridging units include —(CH₂)ₙ—, wherein n is an integer from about 2 to about 20, —CH₂—CH(OH)—CH₂—, —C(O) CH₂ CH₂C(O)—, —CH₂—CH(OH)—O—(CH₂)ₙ—O—CH(OH)—CH₂—, wherein n is 2 to about 4, and —C(O)—(C₆H₂ (COOH)₂)—C(O)—. In preferred embodiments, the bridging unit comprises from about 0.5% to about 20% by weight of the polymer.

Advantageously, cross-linking the polymers renders the polymers non-adsorbable and stable. A “stable” polymer composition, when administered in therapeutically effective amounts, the structure remains intact or otherwise does not decompose to form potentially harmful byproducts.

An “effective amount” is an amount sufficient to effect beneficial or desired clinical results. An effective amount can be administered in one or more administrations. A therapeutically effective dose or amount refers to that amount of the compound sufficient to result in desired treatment.

Desired cross-linked polymer backbones for polyvalent molecule presenters for use in the method of the invention can be prepared via a variety of methods known in the art (Sperling, supra (1994)). For example, a cross-linked polymer can be formed from a first monomer. A second monomer, cross-linker and activating agent are then added to this polymer, swollen in an appropriate solvent, and the second monomer is polymerized and cross-linked in association with the first polymer. In another method, two or more monomers are mixed and simultaneously polymerized and cross-linked by noninterfering reactions. Alternately, two or more polymers are mixed and simultaneously cross-linked by non-interfering reactions. Varying degrees of flexibility of the molecule can be achieved by a variation of one of these methods in which a cross-linking agent for at least one polymer is omitted.

Another method of forming crosslinked polymers involves mixing at least one monomer, at least one pre-formed non-cross-linked polymer and a cross-linking agent for each, and simultaneously polymerizing the monomer(s) and cross-linking via noninterfering reactions.

The monomer can be polymerized by methods known in the art, for example, via an addition process or a condensation process. In one embodiment, the monomer is polymerized via a free-radical process, and the reaction mixture preferably further comprises a free-radical initiator, such as a free radical initiator selected from among those which are well known in the art of polymer chemistry. Suitable free-radical initiators include azobis(isobutyronitrile), azobis(4-cyanovaleric acid), azobis(amidinopropane) dihydrochloride, potassium persulfate, ammonium persulfate and potassium hydrogenpersulfate. The free radical initiator is preferably present in the reaction mixture in an amount ranging from about 0.1 mole percent to about 5 mole percent relative to the monomer.

The choice of cross-linking agents depends upon the identity of the polymers to be cross-linked. Preferably, each polymer is cross-linked via different mechanisms, thereby ensuring that each polymer is cross-linked independently of the other(s). A polymer can be cross-linked, for example, by including a multifunctional co-monomer as the cross-linking agent in the reaction mixture. A multifunctional monomer can be incorporated into two or more growing polymer chains, thereby cross-linking the chains. Suitable multifunctional co-monomers include those discussed above. The amount of cross-linking agent added to the reaction mixture is, generally, between 0.5% and 25% by weight relative to the combined weight of the polymer and the cross-linking agent, and preferably from about 1% to about 10% by weight.

Polymers which comprise primary, secondary or tertiary amino groups can be cross-linked using a co-monomer as discussed above. Such polymers can also be crosslinked subsequent to polymerization by reacting the polymer with one or more crosslinking agents having two or more functional groups, such as electrophilic groups, which react with amine groups to form a covalent bond. Cross-linking in this case can occur, for example, via nucleophilic attack of the amino groups on the electrophilic groups. Suitable cross-linking agents of this type include compounds having two or more groups selected from among acyl chloride, epoxide, and alkyl-X, wherein X is a leaving group, such as a halo, tosyl or mesyl group. Examples of such compounds include epichlorohydrin, succinyl dichloride, butanedioldiglycidyl ether, ethanedioldiglycidyl ether, α,Ω-polyethyleneglycoldiglycidyl ether, pyromellitic dianhydride and dihaloalkanes.

Polymer backbones which are suitable for the present invention include backbones with low intrinsic toxicity.

Other preferred polymer backbones are polysaccharides. Generally, the polysaccharides used to prepare such polymers can be comprised of, for example, glycosyl units connected by glycosidic linkages. These polysaccharides have one reducing end-group. They can be linear or branched, and they may be composed of a single type glycosyl unit or they may be composed of two or more different types of glycosyl units. Other polysaccharides may include, dextran, hydrolyzed dextran, starches, hydrolyzed starches, maltodextrins, cellulose, hydrolyzed cellulose.

In a most preferred embodiment, dextran is used as a polymer backbone due to the hydrophilicity of the polymer, which leads to favorable excretion of conjugates containing the same. Other advantages of using dextran polymers are that such polymers are substantially non-toxic and non-immunogenic, that they are commercially available in a variety of sizes and that they are easy to conjugate to other relevant molecules. Also, dextran-linked conjugates exhibit advantages when non-target sites are accessible to dextranase, an enzyme capable of cleaving dextran polymers into smaller units while non-target sites are not so accessible.

The standard procedure for the introduction of amine groups into dextran has been to first cleave the sugar rings to form polyaldehyde-dextran. The second step is to react the cleaved rings with a diamine such as ethylenediamine or 1,3-diaminopropane to form a Schiff's base complex. The Schiff's base is then stabilized by reduction with sodium borohydride, thus forming the “aminodextran” compounds.

An alternative method of producing aminodextrans is by carboxymethylation of sugar residue hydroxyl groups in chloroacetic acid, followed by carbodiimide coupling of a diamine such as ethylenediamine. M. Brunswick et al., J. Immunol. 140:3364-3372 (1988) and P. K. A. Mongini et al., J. Immunol. 148:3892-3902 (1992) used this method to produce an aminodextran having about one amine group per sixty-seven glucose residues.

A preferred method of producing dextrans to which peptide units are attached, herein referred to as aminodextran, can be prepared by partial cleavage and oxidation of the glucopyranose rings in dextran to give aldehyde flnctional groups, coupling of the aldehyde groups with peptide units of the present invention to form Schiff base linkages and reduction of the Schiff base linkages to form stable carbon-nitrogen bonds. In a typical procedure, 20 g of dextran are dissolved in 150 ml of 50 mM potassium acetate buffer, pH 6.5. A solution of 2.14 g of sodium periodate in 25 ml of distilled water is added dropwise to the dextran over about 10 minutes using vigorous magnetic mixing. The resulting solution is stirred at room temperature, 15° C.-27° C., for about 1.5 hours and then dialyzed against distilled water. 20 ml of peptide units are mixed with 20 ml of distilled water, cooled in an ice bath, vigorously stirred and pH adjusted from about 11.5 to about 8.7 over about 15 minutes by the addition of glacial acetic acid. Typically, 15-20 ml of glacial acetic acid is used. The dialyzed dextran solution is added dropwise over about 15-20 minutes to the chilled dismine solution. After the addition is completed, the resulting solution is stirred at room temperature for about 2.25 hours. A reducing solution of 0.8 g sodium borohydride in 10 ml of 0.1 mM sodium hydroxide is added to the dextran reaction mixture at room temperature over about 15 minutes. The reaction mixture is stirred during the borohydride addition to expel most of the effervescence. The crude aminodextran solution is exhaustively dialyzed against distilled water until the conductivity of the effluent was 3-4 μmho/cm. The dialyzed solution is then filtered through a 0.2 μm filter and freeze-dried over 24 hours in a model TDS-00030-A, Dura-Dry™ microprocessor controlled freeze-dryer (FTS Systems, Inc.) to produce 4.25 g of flaky, pale yellow crystals in 21% yield.

A most preferred method for producing dextran backbones linked to active agents, for example, peptide units, oligonucleotides, proteins, enzymes, nucleic acids, polynucleotides, and the like, and derivatives of such compounds, are disclosed in the examples which follow.

A desired polymer may also be water soluble. The water soluble polymer may be selected from the group consisting of, for example, polyethylene glycol, copolymers of ethylene glycol/propylene glycol, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co- polymers, polyoxyethylated polyols and polyvinyl alcohol. Polyethylene glycol propionaldenhyde may have advantages in manufacturing due to its stability in water.

The polymer may be of any molecular weight, and may be branched or unbranched. For polyethylene glycol, the preferred molecular weight is between about 2 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

The number of polymer molecules so attached may vary, and one skilled in the art will be able to ascertain the effect on function. One may mono-derivatize, or may provide for a di-, tri-, tetra- or some combination of derivatization, with the same or different chemical moieties (e.g., polymers, such as different weights of polyethylene glycols). The proportion of polymer molecules to component or components molecules will vary, as will their concentrations in the reaction mixture. In general, the optimum ratio (in terms of efficiency of reaction in that there is no excess unreacted component or components and polymer) will be determined by factors such as the desired degree of derivatization (e.g., mono, di-, tri-, etc.), the molecular weight of the polymer selected, whether the polymer is branched or unbranched, and the reaction conditions.

In another preferred embodiment, the polymeric backbone is comprised of poly(ethylene glycol. Covalent attachment of the hydrophilic polymer poly(ethylene glycol) (“PEG”), also known as poly(ethylene oxide) (“PEO”), to molecules and surfaces has important applications, including in biotechnology and medicine. In its most common form, PEG is a linear polymer having hydroxyl groups at each terminus:

HO—CH₂—CH₂O(CH₂CH₂O)ₙCH₂CH₂—OH

This formula can be represented in brief as HO—PEG—OH where it is understood that —PEG—represents the following structural unit:

—CH₂CH₂O(CH₂CH₂O)ₙCH₂CH₂—

PEG is commonly used as methoxy-poly(ethylene glycol), or niPEG in brief, in which one terminus is the relatively inert methoxy group, while the other terminus is a hydroxyl group subject to ready chemical modification:

CH₃O—(CH₂CH₂O)ₙCH₂CH₂—OH

Similarly, other alkoxy groups such as benzyloxy and tert-butoxy can be substituted for methoxy in the above formula.

Branched PEGs are also preferred. The branched forms can be prepared by addition of ethylene oxide to various polyols, including glycerol, pentaerythritol and sorbitol. Branched PEGs can be represented as Q(—PEG—OH)ₙ in which Q represents a central core molecule such as pentaerythritol or glycerol, and n represents the number of arms which can range from three to a hundred or more. The hydroxyl groups are readily subject to chemical modification.

The copolymers of ethylene oxide and propylene oxide are closely related to PEG in their chemistry, and they can be substituted for PEG in many of its applications.

HO—CH₂CHRO(CH₂CHRO)₂CH₂CHR—OH

wherein R═H and CH₃; n typically ranges from approximately 10 to 2000.

PEG is a useful polymer having the property of water solubility as well as solubility in many organic solvents. PEG is also non-toxic and non-immunogenic. When PEG is chemically attached to a water insoluble compound, the resulting conjugate generally is water soluble as well as soluble in many organic solvents. When the molecule to which PEG is attached is biologically active, such as a drug, this activity is commonly retained after attachment of PEG and the conjugate may display altered pharmacokinetics. For example, it has been demonstrated that the water insoluble antimalarial, artemisinin, becomes water soluble and exhibits increased antimalarial activity when coupled to PEG. See Bentley et al., Polymer Preprints, 38(1):584 (1997).

U.S. Pat. No. 4,179,337 to Davis et al. discloses that proteins coupled to PEG have enhanced blood circulation lifetime because of reduced kidney clearance and reduced immunogenicity. The lack of toxicity of the polymer and its rapid clearance from the body are advantageous for pharmaceutical applications.

In another preferred embodiment, PEG backbones are prepared having an aldehyde hydrate moiety and reacting the activated PEG directly with a substance containing an amine group without having isolated the activated PEG. An activated PEG having an aldehyde hydrate moiety can be prepared in situ by first linking a PEG polymer with a functional group that can be converted to an aldehyde hydrate moiety, and then hydrolyzing the resulting polymer at an acidic pH. The suitable functional group may have a formula of:

—(CH₂)ₙCH(XR)₂

wherein n is a number of from 1 to 6, X is oxygen O or sulfur S, and R is an alkyl group. The two R groups can be linked or not linked. The linkage between the moiety and the polymer is hydrolytically stable. Typically, the functional group is an acetalaldehyde diethyl acetal moiety or propionaldehyde diethyl acetal moiety, in which n is 1 or 2, respectively.

A substance to be conjugated is added to the reaction mixture, containing the activated polymer having an aldehyde hydrate moiety. The activated PEG polymer in the reaction mixture can readily react with the added substance by reductive amination between the aldehyde hydrate moiety and an amine group in the substance in the presence of a reducing agent.

In other preferred embodiments, in place of the linear PEG polymers, a variety of other polymer forms can be conjugated to an amine-containing substance. Examples of suitable polymer forms include but are not limited to linear or branched or dendritic or star structures, degradable structures, hydrogel forming structures, and others. Other suitable polymers include poly(vinyl alcohol) (“PVA”); other poly(alkylene oxides) such as poly(propylene glycol) (“PPG”) and the like; and poly(oxyethylated polyols) such as poly(oxyethylated glycerol), poly(oxyethylated sorbitol), and poly(oxyethylated glucose); poly(olefinic alcohols); poly(acryloyl morpholine); poly(vinyl pyrrolidone); poly(oxazoline); poly(hydoxyethyl methacrylate, and dextran, and the like.

Amine-containing substances suitable for modification using the method of this invention may include a variety of biomaterials such as peptides, proteins, polysaccharides, oligonucleotides, and the like. Particularly, many drug molecules or carriers are suitable for conjugation.

A poly(ethylene glycol) PEG molecule or a PEG derivative is used as the hydrophilic polymer for conjugation. The starting PEG polymer molecule has at least one hydroxyl moiety, —OH, that is available to participate in chemical reactions and is considered to be an “active” hydroxyl moiety. The PEG molecule can have multiple active hydroxyl moieties available for chemical reaction, as is explained below. These active hydroxyl moieties are in fact usually nonreactive with biological materials, and the first step in the synthesis is to prepare a PEG having a more reactive moiety.

The terms “group,” “functional group,” “moiety,” “active moiety,” “reactive site,” and “radical” are somewhat synonymous in the chemical arts and are used in the art and herein to refer to distinct, definable portions or units of a molecule and to units that perform some function or activity and are reactive with other molecules or portions of molecules. In this sense a protein or a protein residue can be considered a molecule or as a functional group or moiety when coupled to a polymer.

The term “PEG” is used in the art and herein to describe any of several condensation polymers of ethylene glycol having the general formula represented by the structure H(OCH₂ CH₂)ₙOH. PEG is also known as polyoxyethylene, polyethylene oxide, polyglycol, and polyether glycol. PEG can be prepared as copolymers of ethylene oxide and many other monomers.

Poly(ethylene glycol) is used in biological applications because it has properties that are highly desirable and is generally approved for biological or biotechnical applications. PEG typically is clear, colorless, odorless, soluble in water, stable to heat, inert to many chemical agents, does not hydrolyze or deteriorate, and is nontoxic. Poly(ethylene glycol) is considered to be biocompatible, which is to say that PEG is capable of coexistence with living tissues or organisms without causing harm. More specifically, PEG is not immunogenic, which is to say that PEG does not tend to produce an immune response in the body. When attached to a moiety having some desirable function in the body, the PEG tends to mask the moiety and can reduce or eliminate any immune response so that an organism can tolerate the presence of the moiety. Accordingly, the PEG polymers of the invention should be substantially non-toxic and should not tend substantially to produce an immune response or cause clotting or other undesirable effects.

The polyethylene glycol molecules (or other chemical moieties) should be attached to the component or components with consideration of effects on functional or antigenic domains of the protein. There are a number of attachment methods available to those skilled in the art, e.g., EP 0 401 384 herein incorporated by reference, see also Malik et al., 1992, Exp. Hematol. 20:1028-1035. For example, polyethylene glycol may be covalently bound through amino acid residues via a reactive group, such as, a free amino or carboxyl group. Reactive groups are those to which an activated polyethylene glycol molecule may be bound. The amino acid residues having a free amino group include lysine residues and the N- terminal amino acid residues; those having a free carboxyl group include aspartic acid residues glutamic acid residues and the C-terminal amino acid residue. Sulfhydryl groups may also be used as a reactive group for attaching the polyethylene glycol molecule(s).

Preferred for therapeutic purposes is attachment at an amino group, such as attachment at the N-terminus or lysine group. One may specifically desire N-terminally chemically modified protein. Using polyethylene glycol as an illustration of the present compositions, one may select from a variety of polyethylene glycol molecules (by molecular weight, branching, etc.), the proportion of polyethylene glycol molecules to protein (or peptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective N-terminal chemically modification may be accomplished by reductive alkylation which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved. For example, one may selectively N-terminally pegylate the protein by performing the reaction at a pH which allows one to take advantage of the PKₐ differences between the ε-amino groups of the lysine residues and that of the α-amino group of the N-terminal residue of the protein. By such selective derivatization, attachment of a water soluble polymer to a protein is controlled: the conjugation with the polymer takes place predominantly at the N-terminus of the protein and no significant modification of other reactive groups, such as the lysine side chain amino groups, occurs. Using reductive alkylation, the water soluble polymer may be of the type described above, and should have a single reactive aldehyde for coupling to the protein. Polyethylene glycol proprionaldehyde, containing a single reactive aldehyde, may be used.

An illustrative example of a method of conjugating is to link to the PEG polymer, a moiety that can be conveniently converted or hydrolyzed to an aldehyde hydrate group. This moiety should not be an aldehyde group. In a preferred embodiment of the present invention, the moiety to be linked to the PEG polymer has a formula of

—(CH₂)ₙCH(XR)₂

wherein n is a number of from 1 to 6, X is the atom of O or S, and R is an alkyl group. The two R groups can be linked together or not linked. The linkage between the moiety and the polymer is hydrolytically stable.

As indicated by the formula, the moiety to be linked to PEG polymer can be a variety of groups, e.g., diethyl acetal group (when n=1, X is oxygen atom, R is an alkyl group with two carbons), propionaldehyde diethyl acetal group (n=2, X is oxygen atom, R is an alkyl group with two carbons). Preferably, the moiety is diethyl acetal group.

The linking can be done by reacting a PEG polymer having at least one hydroxyl group with a halide substituted compound having a formula of

Halide-(CH₂)ₙCH(XR)₂

wherein n is a number of from 1 to 6, X is the atom of O or S, and R is an alkyl group. The two R groups can be linked or not linked to each other. The reaction is completed in the presence of for example, sodium hydroxide.

The second step is to convert the above polymer precursor to an activated organic polymer having an active aldehyde hydrate moiety. This hydrolysis is done conveniently in situ in an aqueous solution at an acidic pH. Without being bound by any theory, it is believed that the conversion is a result of the reaction of the moiety in the precursor polymer with water. An acidic pH in the reaction mixture can be generated by adding acids to the reaction which is generally known in the art. For example, acetic acid, phosphoric acid, trifluoroacetic acid are all suitable. The reaction time required for the conversion can vary with temperature and the acid used. Typically, the time required for complete hydrolysis is shorter when a higher temperature is maintained. In addition, lower pH leads to shorter duration required for complete hydrolysis.

A substantially complete conversion from the polymer precursor to the aldehyde hydrate polymer can be achieved in accordance with this invention. Spectroscopic tests can be performed to analyze the components in the reaction mixture after the conversion is completed. Substantially 100% conversion can be achieved with no detectable aldehyde derivative of the polymer present, particularly for the acetaldehyde.

The resulting activated organic polymer having an active aldehyde hydrate moiety can be readily used to react with a substance by reductive amination. In the reaction, the aldehyde hydrate moiety acts as a functional group and reacts with the amine group in the substance. In accordance with the present invention, in the conjugation step, the substance to which the PEG polymer to be conjugated is added to the reaction mixture directly. In addition, a reductive agent must be added to the reaction. An exemplary example of such a reductive agent is sodium cyanoborohydride(NaCNBH₃). Specifically, the conjugation is by reductive amination. Thus, the substance must contain an amine group on its surface or particle. The substance can be selected from, e.g., proteins, peptides, oligonucleotides, polysaccharides and small drug molecules. Broadly speaking, any material having a reactive amine group accessible to the activated polymer having an aldehyde hydrate group can be used in the present invention. Most preferred are peptide units that inhibit assembly of toxins derived from infectious disease causing agents.

The PEG can be substituted or unsubstituted so long as at least one reactive site is available for conversion into an aldehyde hydrate moiety. PEG typically has average molecular weights of from 200 to 100,000 and its biological properties can vary with molecular weight and depending on the degree of branching and substitution, so not all of these derivatives may be useful for biological or biotechnical applications. For many biological and biotechnical applications, substantially linear, straight-chain PEG acetaldehyde hydrate is useful, substantially unsubstituted except for the acetaldehyde hydrate moieties and, where desired, other additional functional groups. The PEG can be capped on one end with a relatively nonreactive moiety such as a moiety selected from the group consisting of alkyl moieties, typically methyl, benzyl moieties and aryl moieties. The capped form can be useful, for example, if it is desirable simply to attach the polymer chains at various amine sites along a protein chain. Attachment of PEG molecules to a biologically active molecule such as a protein or other pharmaceutical or to a surface is sometimes referred to as “PEGylation.”

A linear PEG with active hydroxyls at each end can be activated at each end to have an aldehyde hydrate group at each end. This type of activated PEG is said to be homobifunctional. The bifunctional structure, PEG bis aldehyde hydrate, for example, a dumbbell structure and can be used, for example, as a linker or spacer to attach a biologically active molecule to a surface or to attach more than one such biologically active molecule to the PEG molecule. In addition, bifunctional activated PEG can be used to cross-link biological materials such proteins, aminopolysacchrides such as chitosan to form hydrogel.

Another form of activated PEG aldehyde hydrate is dendritic activated PEG in which multiple arms of PEG are attached to a central core structure. Dendritic PEG structures can be highly branched and are commonly known as “star” molecules. Examples of suitable molecules for the core include but not limited to glycerol, lysine, pentaerythritol. A “star” molecule can be represented by the formula of Q[poly]_{y}.

Wherein Q is a branching core moiety and y is from 2 to about 100. Star molecules are generally described in U.S. Pat. No. 5,171,264 to Merrill, the contents of which are incorporated herein by reference. The aldehyde hydrate moiety can be used to provide an active, functional group on the end of the PEG chain extending from the core and as a linker for joining a functional group to the star molecule arms. Additionally, the aldehyde hydrate moiety can also be linked directly to the core molecule having PEG chains extending from the core. One example of such a dendritic activated PEG has a formula of

[RO—(CH₂CH₂O)ₘCH₂CH₂—O—CH₂]₂CH—O—(CH₂)ₙCH(OH)₂

wherein R is H, alkyl, benzyl, or aryl; m ranges from about 5 to about 3000, n ranges from 1 to 6.

PEG aldehyde hydrate and its derivatives can be used for attachment directly to surfaces and molecules having an amine moiety. However, a heterobifuinctional PEG derivative having a aldehyde hydrate moiety on one terminus and a different functional moiety on the opposite terminus group can be attached by the different moiety to a surface or molecule. When substituted with one of the other active moieties, the heterobifunctional PEG dumbbell structure can be used, for example, to carry a protein or other biologically active molecule by amine linkages on one end and by another linkage on the other end, such as sulfone linkage, to produce a molecule having two different activities. A heterobifuinctional PEG having an amine specific moiety on one end and a sulfone moiety on the other end could be attached to both cysteine and lysine fractions of proteins. A stable sulfone linkage can be achieved and then the hydrolytically stable unreacted aldehyde hydrate moiety is available for subsequent amine-specific reactions as desired.

It should be apparent to the skilled artisan that the dumbbell structures discussed above could be used to carry a wide variety of substituents and combinations of substituents. Pharmaceuticals such as aspirin, vitamins, penicillin, and others too numerous to mention; polypeptides or proteins and protein fragments of various functionalities and molecular weights; cells of various types. As used herein, the term “protein” should be understood to include peptides and polypeptides, which are polymers of amino acids. “Biopolymer” should be taken as a descriptive word for compounds of biological origin, such as proteins, enzymes, nucleic acids, polynucleotides, peptides and the like, and derivatives of such compounds.

Preferred biomolecules for attachment to the PEG backbone are compounds of biological origin, such as proteins, enzymes, nucleic acids, polynucleotides, peptides and the like, and derivatives of such compounds. Most preferred attachments to the PEG polymeric backbone are peptide units that inhibit assembly of toxins, for example anthrax toxin.

One straight chain activated PEG derivative for biological and biotechnical applications has the basic structure of

Z—O—(CH₂CH₂O(CH₂CH₂O)ₘ(CH₂)ₙCH(OH)₂

The PEG monomer —OCH₂CH₂— preferably is substantially unsubstituted and unbranched along the polymer backbone. The letter “m” can equal from about 5 to 3,000. A more typical range is from about 5 to 2,200, which corresponds to a molecular weight of from about 220 to 100,000. Still more typical is a range of from about 34 to 1,100, which corresponds to a molecular weight range of from about 1,500 to 50,000. Most applications will be accomplished with molecular weights in the neighborhood of 2,000 to 5,000, which corresponds to a value of m of from about 45 to 110.

Suitably, n ranges from 1 to 6. Z is selected from the group consisting of hydrogen, alkyl groups, benzyl groups and aryl groups.

The active polymer derivatives are water soluble and produce water soluble stable linkages with amine groups. The derivatives are considered infinitely soluble in water or as approaching infinite solubility and can enable otherwise insoluble molecules to pass into solution when conjugated with the derivative.

Other water soluble polymers that may be used in the present invention and are believed to be suitable for similar modification and activation with an active aldehyde hydrate moiety, include, for example, poly(vinyl alcohol) (“PVA”); other poly(alkylene oxides) such as poly(propylene glycol) (“PPG”) and the like; and poly(oxyethylated polyols) such as poly(oxyethylated glycerol), poly(oxyethylated sorbitol), and poly(oxyethylated glucose); poly(olefinic alcohols); poly(acryloyl morpholine); poly(vinyl pyrrolidone); poly(oxazoline); poly(hydoxyethyl methacrylate, and dextran, and the like. The polymers can be homopolymers or random or block copolymers and terpolymers based on the monomers of the above polymers, straight chain or branched, or substituted or unsubstituted similar to PEG, but having at least one active site available for reaction to form the aldehyde hydrate moiety.

Other suitable backbones include, for example, chitin/chitosan, cellulose; polypeptides comprising natural or synthetic amino acid residues such as, for example, polylysine, polyamides, polyglutamic acid, and polyaspartic acid; oligonucleotides such as, for example, DNA and RNA; polycarbohydrates or polysaccharides such as, for example, polyamylose, polyfuranosides, polypyranosides, carboxymethylamylose, and dextrans; polystyrenes such as, for example, chloromethylated polystyrene and bromomethylated polystyrene; polyacrylamides such as, for example, polyacrylamide hydrazide; polyacids such as, for example, polyacrylic acid; polyols such as, for example, polyvinyl alcohol; polyvinyls such as, for example, polyvinyl chloride and polyvinyl bromide; polyesters; polyurethanes; polyolefins; polyethers; and the like as well as other monomeric, polymeric or oligomeric materials containing reactive functional groups along the length of their chain which can be substituted with a phosphorothioate monoester group.

Synthesis of the crosslinking and conjugating agents can generally be accomplished by functionalizing a monomer, polymer or oligomer with a phosphorothioate monoester functionality using methodologies which are well known to those skilled in the art. Backbones having, for example, carboxylate functionalities or hydroxyl functionalities such as, for example, polyglutamic acid, polyacrylic acids, carboxymethyl amylose and the like, can be functionalized with phosphorothioate monoester by (i) activating carboxylate or hydroxyl functionalities with a suitable electrophilic activator such as, for example, (1-ethyl 3-(3-dimethylaminopropyl) carbodiimide (EDAC) or bromoacetic acid followed by EDAC and (ii) reacting the so-formed activated esters with cysteamine-S-phosphate. A most preferred backbone is dextran poly(phosphorothioate). Backbone polymers having haloalkyl styrene residues can be functionalized with a phosphorothioate monoester by reacting a para or ortho phenyl alkyl halide with sodium thiophosphate (Na₃ SPO₃).

The polyvalent molecule presenter can be administered intravenously or intramuscularly by a suitable mechanical device, such as hypodermic needle and syringe, air gun injection devises, inhalation devices, etc., at a dosage of about 1 mg/kg/day to about 10 g/kg/day depending upon the individual patient. The polyvalent molecule presenter of the present invention can also be administered orally to a patient in a dosage of about 1 mg/kg/day to about 10 g/kg/day; the particular dosage will depend on the individual patient (e.g., the patient's weight and the extent of bile salt removal required). The polymer can be administered either in hydrated or dehydrated form, and can be flavored or added to a food or drink, if desired, to enhance patient acceptance.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin Remington's Pharm. Sci., 15th Ed. (Mack Publ. Co., Easton (1975)). Pharmaceutically acceptable carriers are sterile and pyrogen-free.

Examples of suitable forms for administration include pills, tablets, capsules, and powders (i.e. for sprinkling on food). The pill, tablet, capsule or powder can be coated with a substance capable of protecting the composition from the gastric acid in the patient's stomach for a period of time sufficient for the composition to pass undisintegrated into the patient's small intestine. The polymer can be administered alone or in combination with a pharmaceutically acceptable carrier substance, e.g., magnesium carbonate or lactose.

The polyvalent molecule presenter can be administered intramuscularly, intravenously, intrapulmonary, orally, rectally or by any additional means which can deliver the polymer to mucosal surfaces and circulating body fluids. The polyvalent molecule presenter can be administered orally, rectally or by any additional means which can deliver the polymer to the intestinal tract. The quantity of an individual polyvalent molecule presenter to be administered will be determined on an individual basis and will be determined, at least in part, by consideration of the individual's size, the severity of symptoms to be treated and the result sought.

The polyvalent molecule presenter can be administered as a solid or in solution, for example, in aqueous or buffered aqueous solution. The polyvalent molecule presenter can be administered alone or in a pharmaceutical composition comprising the polyvalent molecule presenter, an acceptable carrier or diluent and, optionally, one or more additional drugs.

As used herein, “patient” refers to any animal or mammal, and includes but is not limited to, domestic animals, sports animals, primates and humans; more particularly, the term refers to humans.

As used herein, the singular forms “a”, “an” and “the” include plural referents unless the context clearly dictates otherwise.

The following non-limiting examples are illustrative of the invention. All documents mentioned herein are incorporated herein by reference in their entirety.

### EXAMPLES

Materials and Methods

Phage-Display Selection and ELISA.

Purified heptamer, 2 μg, was coated in Maxisorp tubes (Nunc) in phosphate buffered saline (PBS) overnight at 4° C. The tubes were blocked with PBS-2% bovine serum albumin (BSA) at 37° C. for 2 h and washed with PBS. M13 bacteriophages (1.5×10¹¹ pfu), present in a library displaying 12-amino acid, 7-amino acid or cysteineconstrained 7-amino acid peptides fused to the N-terminus of the pill protein (PhD12, PhD7, PhDC7C, New England Biolabs), were allowed to bind the heptamer in PBS-0.1% Tween 20 at room temperature for 60 min in round 1, 30 min in round 2, and 5 min in round 3 (step 1). After binding, the wells were washed eight times (step 2). Purified intact PA (15 μg in PBS) was added at room temperature for 1 h (step3) and then the remaining phages were eluted with 40 μg of heptamer in PBS at room temperature (step4) for 60 min in round 1 and overnight in rounds 2 and 3. The selection was repeated three times, and the eluted phages amplified between rounds.

For ELISA, 1 μg of protein (PA63 heptamer, black bars, intact PA, gray bars) was coated in wells of a 96-well Maxisorp plate (Nunc) in PBS overnight at 4° C. The plate was blocked for 2 h at 37° C. with PBS-2% BSA. Phages (10⁸ pfa in PBS), displaying different peptides (P1-4), or the unselected library as a negative control, were allowed to bind to the coated surface in the presence or absence of 10 μM LF_{N} (striped bars). Bound phages were revealed using a monoclonal anti-M13 antibody coupled to horseradish peroxidase (Pharmacia). The enzymatic activity was assayed by oxidation of 3,3′,5,5′-tetramethylbenzidine, measured by absorbance at 450 mn. ELISA were performed in duplicate and repeated twice.

Methods for Testing the Potency of PVI

i) Cell Binding of Radioactively Labelled LF_{N}.

Confluent CHO cells in a 24-well plate were incubated for 1 h on ice in HAM medium buffered with 20 mM Hepes, pH 7.4, in the presence of 2×10⁻⁸ M PA cleaved by trypsin as described elsewhere¹⁹. LF_{N} was labeled with ³⁵S-methionine by in vitro coupled transcription and translation, as described²⁴. After one wash with cold PBS, radioactive LF_{N} was added for 1 h to the cells on ice in the presence of various amounts of LF_{N}, PVI, underivatized polymer, or monomeric peptide. The cells were then washed and lysed, and the radioactivity in the lysate was measured. The background of LF_{N} bound to cells in absence of PA was subtracted and was less than 5% of control. The inhibition of LF_{N} binding is expressed as the percentage of radioactivity of the control (radioactivity bound on cells incubated without inhibitor) that was not bound. The results are the mean±standard error on the mean (s.e.m.) of three independent experiments.

ii) Cytotoxicity Assay of LF_{N}DTA.

Confluent CHO cells in a 96-well plate were incubated with 10⁻⁹M PA and 2×10⁻¹¹M LF_{N}DTA with various amounts of LF_{N}, PVI, backbone or peptide. The cells were incubated for 4 h at 37° C. and then protein synthesis was assayed by monitoring ³H leucine incorporation in cellular proteins. The amount of radioactivity incorporated in the absence of inhibitor was less than 2% of control. The inhibition of toxicity is expressed as the percentage of radioactivity of the control (radioactivity recovered from cells incubated without LF_{N}DTA). Each experiment was done in duplicate. The results are the mean±s.e.m. of three independent experiments.

iii) Rat Intoxication

Purified PA (40 g) and LF (8 μg) diluted in PBS were mixed with: PBS, a mixture of 125 μg of peptide and 125 μg of polymer backbone, 72 μg or 450 μg of PVI. Fisher 344 rats (250-300 g, Harlan Laboratories) were injected intravenously in the dorsal vein of the penis after anesthesia by intraperitoneal injection of ketamine and acepromazine. Four rats per group were injected with the different mixtures, and the appearance of symptoms of intoxication monitored. When the symptoms were obvious, the rats were sacrificed to avoid unnecessary distress. In post challenge protection experiments, four rats were injected with PA and LF diluted in PBS. Three to four minutes afterwards, a new syringe was used to inject at the same site PVI diluted in PBS.

Methods for Preparation of Poly-Glutamic Acid-Peptide Inhibitors

Polyvalent Inhibitor JJM1:

3 mg of low molecular weight “D” isomer poly-glutamic acid (MW 13,000, Sigma P 5261) was dissolved in 5 ml of water and the pH of the solution adjusted to 4.5 with dilute hydrochloric acid (“PG13 Solution”). A solution of 1-ethyl-3-(3dimethylaminopropyl)carbodiimide (Sigma 1769) was freshly prepared by dissolving 92 mg in 4.5 ml of water (“EDAC Solution”). 1 mg of TYWWLDGAPK peptide was dissolved in 1 ml of water and adjusted to pH 4.5 with diluted hydrochloric acid or sodium hydroxide solution (“Peptide K Solution”). The γ-carboxyl groups of the poly-Dglutamic acid was then activated by addition of 100 μl of EDAC solution to the PG13 solution. The reaction was allowed to proceed at room temperature (about 22° C.) while constantly adjusting the pH to 4.5 with dilute hydrochloric acid. After 10 minutes another 100 μl of EDAC solution was added to the reaction vessel and the reaction allowed to continue at room temperature while constantly adjusting the pH to 4.5 with dilute hydrochloric acid. After 10 more minutes a third 100 μl addition of EDAC solution was added to the reaction vessel and the reaction allowed to continue at room temperature while constantly adjusting the pH to 4.5 with dilute hydrochloric acid. After 2 more minutes 1 ml of Peptide K Solution was added to the reaction vessel. The reaction was allowed to proceed for 5 hours at room temperature. The reaction was then terminated by dialysis (using 6000 MW cut off tubing) against 25 mM sodium acetate buffer at 4° C. for 18 hours followed by dialysis against water for an additional 18 hours at 4° C. Spectrophotometric analysis indicated that approximately one K peptide molecule was coupled per 44 residues of glutamic acid in the final product (designated Polyvalent inhibitor JJM1).

Polyvalent Inhibitor JJM2:

3 mg of high molecular weight “D” isomer polyglutamic acid (MW 38,000, Sigma P 4033) was dissolved in 5 ml of water and the pH of the solution adjusted to 4.5 with dilute hydrochloric acid (“PG38 Solution”). A solution of EDAC was freshly prepared by dissolving 92 mg in 4.5 ml of water (“EDAC Solution”). 1 mg of peptide was dissolved in 1 ml of water and adjusted to pH 4.5 with diluted hydrochloric acid or sodium hydroxide solution (“Peptide K Solution”). The γ-carboxyl groups of the poly-D-glutamic acid was then activated by addition of 100 μl of EDAC solution to the PG38 solution. The reaction was allowed to proceed at room temperature (about 22° C.) while constantly adjusting the pH to 4.5 with dilute hydrochloric acid. After 10 minutes another 100 μl of EDAC solution was added to the reaction vessel and the reaction allowed to continue at room temperature while constantly adjusting the pH to 4.5 with dilute hydrochloric acid. After 10 more minutes a third 100 ul addition of EDAC solution was added to the reaction vessel and the reaction allowed to continue at room temperature while constantly adjusting the pH to 4.5 with dilute hydrochloric acid. After 2 more minutes 1 ml of Peptide K Solution was added to the reaction vessel. The reaction was allowed to proceed for 5 hours at room temperature. The reaction was then terminated by dialysis (using 6000 MW cutoff tubing) against 25 mM sodium acetate buffer at 4° C. for 18 hours followed by dialysis against water for an additional 18 hours at 4° C. Spectrophotometric analysis of the fully dialyzed product indicated that approximately one K peptide molecule was coupled per 42 residues of glutamic acid in the final product (designated Polyvalent inhibitor JJM2).

Polyvalent Inhibitor JJM4:

3 mg of high molecular weight “L” isomer polyglutamic acid (MW 31,700 Sigma P 4761) was dissolved in 5 ml of water and the pH of the solution adjusted to 4.5 with dilute hydrochloric acid (“PG31 Solution”). A solution of EDAC was freshly prepared by dissolving 92 mg in 4.5 ml of water (“EDAC Solution”). 1 mg of peptide was dissolved in 1 ml of water and adjusted to pH 4.5 with diluted hydrochloric acid or sodium hydroxide solution (“Peptide K Solution”). The γ-carboxyl of the poly-L-glutamic acid was then activated by addition of 100 μl of EDAC solution to the PG31 solution. The reaction was allowed to proceed at room temperature (about 22° C.) while constantly adjusting the pH to 4.5 with dilute hydrochloric acid. After 10 minutes another 100 μl of EDAC solution was added to the reaction vessel and the reaction allowed to continue at room temperature while constantly adjusting the pH to 4.5 with dilute hydrochloric acid. After 10 more minutes a third 100 ul addition of EDAC solution was added to the reaction vessel and the reaction allowed to continue at room temperature while constantly adjusting the pH to 4.5 with dilute hydrochloric acid. After 2 more minutes 1 ml of Peptide K Solution was added to the reaction vessel. The reaction was allowed to proceed for 5 hours at room temperature. The reaction was then terminated by dialysis (using 6000 MW cutoff tubing) against 25 mM sodium acetate buffer at 4° C. for 18 hours followed by dialysis against water for an additional 18 hours at 4° C. Spectrophotometric analysis of the fully dialyzed product indicated that approximately one K peptide molecule was coupled per 42 residues of glutamic acid in the final product (designated Polyvalent inhibitor JJM4)

Methods for Preparation of Dextran-Peptide Inhibitors

Conjugate YW3-2:

To a 11.1-mg dextran 40 (avg. M.W. 40 kDa) sample was added 0.85 mL of 20 mM of NaIO₄ in 0.05 M NaAc buffer. The reaction was preceded in the dark at room temperature for 2 hours and then at 4° C. for 12 hours. The mixture was purified on a PD10 column and then lyophilized. 5.9 mg of peptide (TYWWLDGAPK) was mixed with 1.6 mg of oxidized dextran and dissolved in 0.2 mL of 0.05M of Na₂CO₃ solution. After stirring at room temperature for 1 hr, 5 mg of NaBH₃CN was added to the solution. The mixture was stirred for another 12 hours. The product was purified on a PD10 column and lyophilized. The conjugate was analyzed by proton NMR spectroscopy.

Conjugate YW3:

To an 11.1-mg dextran 40 (avg. M.W. 40 kDa) sample was added 0.85 mL of 20 mM of NaIO₄ in 0.05 M NaAc buffer. The reaction was preceded in the dark at room temperature for 2 hrs and then at 4° C. for 12 hours. The mixture was purified on a desalting PD10 column and then lyophilized. 3.8 mg of peptide (TYWWLDGAPK) was mixed with 0.5 mg of oxidized dextran and dissolved in 0.2 mL of 0.05M of Na₂CO₃ solution. After stirring at room temperature for 1 hr, 5 mg of NaBH₃CN was added to the solution. The mixture was stirred for another 12 hours. The product was purified on a PD10 column and lyophilized. The conjugate was analyzed by proton NMR spectroscopy.

### Example 1

Selection of Peptides by Phage-Display

To inhibit activity of anthrax toxin the assembly of PA, LF, and EF into toxic complexes, was interfered with. To develop an inhibitor of this process, phage display⁵ was used to identify peptides that interfered with binding of EF and LF to PA63. The rationale was to block the assembly of toxin with a peptide, binding a surface, specific to the heptamer. Since the heptamer but not PA83, can interact with EF or LF, surfaces specific to the heptamer are thought to be involved in the interaction with EF/LF. Peptides binding these surfaces should compete with LF/EF for binding on the heptamer.

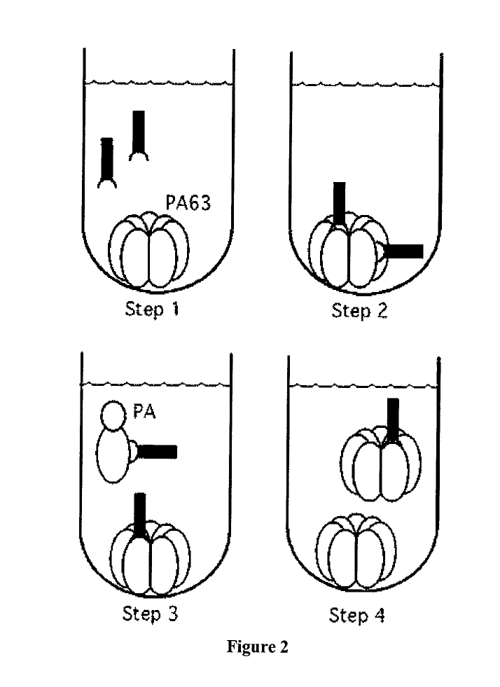

A protocol was devised to select for members of a phage library that bind to PA63 and eliminate those that bind to the uncleaved PA molecule (FIG. 2). This protocol enriched for phages that bind at or near the EF/LF binding site of PA63. PA63 was adsorbed onto a plastic surface and added a library of M13 phages displaying random 12-residue peptides fused to the N-terminus of the pIII protein. After incubation, the surface was washed and then intact PA was added to elute phages that bound to the whole protein. Finally soluble PA63 heptamer was added, and phages that adsorbed to it were recovered.

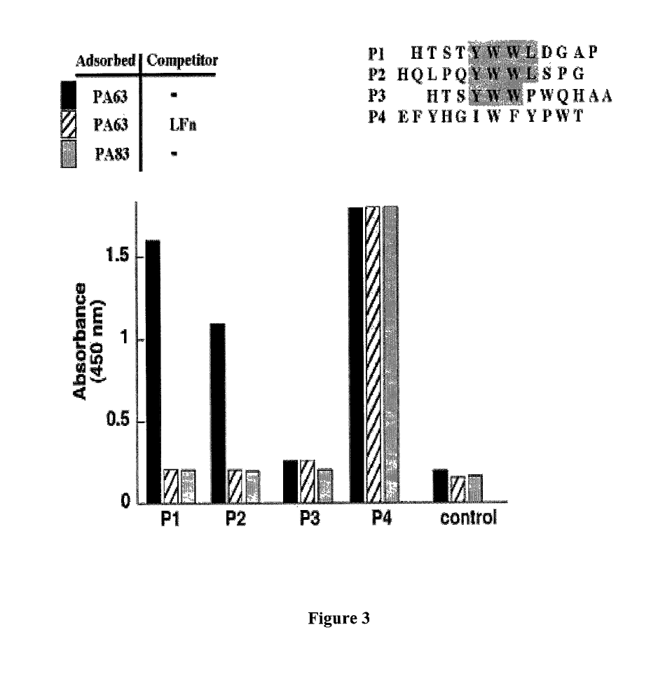

After three rounds of selection (FIG. 3) two phages, P1 and P2, were identified, which could bind on PA63 adsorbed on plastic (black bar) but not on PA83 (gray bar). The binding of these phages could be competed off by adding 10 μM LFn (hatched bar), the domain of LF involved in the interaction with the heptamer. This suggests that these peptides are allowing the phages to bind on a site close to, or structurally related to, the binding site of LFn. By contrast, P4, a phage binding PA83 and the heptamer is not blocked by LFn and yet it does not bind LFn. The peptides displayed by P1 and P2 bear a YWWL motif. This suggests that this motif might be critical in allowing binding, although this sequence can not be found in the “natural” ligands of the PA₆₃ heptamer (LF_{N}, EF_{N} or PA₂₀). A phage displaying a peptide with almost the same motif (P3) did not bind PA63, suggesting that the YWWL motif is the minimal sequence required for binding.

Using the same approach, other libraries of phage displayed peptides were selected and two other phages displaying the sequences HYTYWWL and CWSSFAWYC showed the same properties as P1 and P2 (data not shown). The last peptide did not show the YWWL motif. It was isolated from a library of phages displaying “cysteine-constrained” peptides, peptides that are bordered by two cysteines presumably form a disulfide bond which may force the peptide to adopt a cyclic structure. The hydrophobicity of the sequence isolated is consistent with the hydrophobicity of the YWWL motif. The peculiar conformation of the “cysteine-constrained” peptide might explain why the motif was not isolated again.

The P1 and P2 peptides share the hydrophobic sequence, YWWL; this commonality suggests that this tetrapeptide may play a role in binding to PA63. The sequence YWWL is not present in EF, LF or PA20. The side chains of three contiguous aromatic residues (Y22, Y23, and F24) of the PA20 moiety of native PA do, however, contact the hydrophobic surface of the PA63 moiety. The YWWL sequence may bind to PA63 at this site, which is exposed to the solvent after removal of PA20³.

The P1 dodecapeptide—HTSTYWWLDGAP—was synthesized and found to disrupt the binding of radiolabeled LF_{N} to PA63 on CHO cells. A control peptide, FDLPFTMSTPTP, had no effect. The weak inhibitory activity of the P1 peptide (IC₅₀˜150 μM, see below) precluded its use as an inhibitor in vivo.

The following peptides were synthesized and assayed for their ability to prevent LFn binding on PA63 heptamers formed on the surface of CHO mammalian cells:

HTSTYWWLDGAP

HTSTYWWLDGAPK

HTSTYWWLD

TYWWLDGAP

TYWWLDGAPK

TYWWLSPGK

Of these peptides all but one, HTSTYWWLD, could prevent radiolabeled LF_{N} from binding on the Pa₆₃ heptamer. This suggests that the amino acids coming immediately after the YWWL motif also play a fundamental role in binding. However, no specific amino acid is conserved among the C-terminal residues of the peptides, which might suggest that only a backbone carboxyl is needed to allow binding.

### Example 2

Synthesis of Polyvalent Inhibitors Based on Carbohydrate Backbones

Peptide TYWWLDGAPK, was used in the synthesis of polyvalent molecules based on a backbone of dextran chains of 40 kDa. The resultant molecules YW3 and YW3-2 have different peptide:dextran ratios.

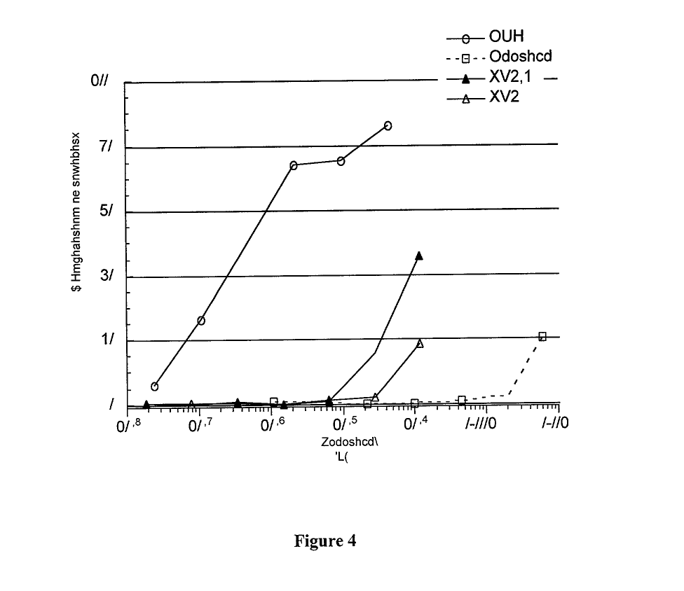

The potency of these various polyvalent molecules was assayed, by testing their ability to inhibit the toxicity of LFn-DTA and PA towards CHO cells (FIG. 4). The dextran based compounds were more effective than peptide alone. These carbohydrate-based backbones increased the potency of the peptide 20 to a 100 fold. While the original acrylamide backbone increased the potency of the peptide almost 10,000 fold it must be noted that these carbohydrate backbones are shorter than the original acrylamide backbone (roughly two fold) and have less peptides displayed per molecule (10 to 4 times less).

### Example 3

Characterization of the LF/EF Binding Site on PA63

Several lines of evidence are yielding an emerging concept of the location and nature of the LF/EF binding site on heptameric PA63. These data come from: (i) directed mutagenesis of PA; (ii) directed mutagenesis of LF_{N} (the N-terminal, PA63-binding domain of LF); (iii) studies on the relationship of oligomerization of PA63 to the formation of the LF/EF site; and (iv) the nature of inhibitory peptides that bind, we believe, at or near the site. The crystallographic structures of native PA, the PA63 prepore, and LF provide a structural framework for this analysis.

(i) Identification of the LF_{N}-binding site on the PA63 heptamer was undertaken after a comparison of PA83 to several PA-like proteins from spore-forming Gram-positive bacteria revealed a stretch of residues that lacked homology, in a domain of high sequence similarity. The surface formed by these residues in PA83 becomes fully exposed upon formation of the PA63 heptamer and was hypothesized to be the LF_{N}-binding site. Alanine-scanning mutagenesis has enabled us to identify a patch of residues within this surface is involved in binding of radioactive LF_{N}. Constructs that contain the substitutions P205A, I207A, and K214A completely eliminated binding of LF_{N}. Constructs that contain the substitutions D195A and H211A had 30% of wild-type binding, while constructs that contain E190A, K213A, and K218A had 60% of wild-type binding. All alanine mutants were able to form SDS-resistant heptamer on the surface of CHO cells indicating the substitutions do not prevent heptamer formation. The three-dimensional structure of the PA63 heptamer indicates residues D195, P205, I207, H211 and K214 form a surface-exposed cluster flanked by residues E190, K213, and K218. Additional mutagenesis studies are underway to extend the cluster and define the border of the LFn-binding site on the PA63 heptamer.

(ii) Efforts to identify the PA-binding site of LF_{N} have stemmed from an analysis of the conserved residues between EF and LF and their location on the LF three-dimensional structure. One surface of LF_{N} contained a concentration of conserved residues and was hypothesized to be the PA-binding site. Using mutagenesis and binding studies of radioactive LF_{N} to PA on cells, the binding site has been mapped to a small patch within this surface. Constructs of LF_{N} containing the single mutant Y236A or the double mutant D182A/D184A do not bind PA. These three residues are clustered together on the surface of the structure and are immediately surrounded by residues L188 and Y223. Constructs containing the single mutants L188A and Y223A show a reduction in binding. To date, all alanine mutations made in residues outside of this sphere of binding have shown no effect on binding although there are a few more left to test. The corresponding mutations are being made in EF and the single mutants of D182A and D184A are being tested individually. The PA-binding site of LF_{N} has amino acids similar to those observed in the consensus sequence of the peptide inhibitors and could be used for rationally improving the binding properties of these inhibitors.

(iii) The role of oligomerization of PA63 in the intoxication process, was studied by constructing two mutants that do not oligomerize. The first mutant has a lysine residue substituted for an aspartate at position 512. The second contains mutations that change amino acid 199 from lysine to glutamate, amino acid 468 from arginine to alanine, and amino acid 470 from arginine to aspartate. Each mutant has one wild-type and one mutated oligomerization surface; the mutants differ by which of their surfaces is competent for oligomerization and which is defective. Dimeric PA63 can be formed by mixing the two mutants on cells, because their complementary wild-type oligomerization surfaces interact and their mutant surfaces prevent further oligomerization. We have found that oligomerization-defective mutants by themselves do not associate stably with the PA-binding domain of lethal factor (LF_{N}). Dimeric PA63 does bind LF_{N}, but can not mediate its translocation. Thus we believe that monomeric PA63 does not contain a high-affinity site for LF/EF, and that such a site is generated (or stabilized) only upon the interaction of two PA63 monomers in the process of assembly of the heptamer.

(iv) Using phage display, peptides binding specifically the PA₆₃ heptamer, and not PA₈₃, could be selected. The sequences of these phage-displayed peptides are: HQLPQYWWLSPG; HTSTYWWLDGAP(*) (from which the following peptides were derived: HTSTYWWLDGAPK (*), TYWWLDGAP (*),TYWWLDGAPK (*)); TYWWLSPGK (*); HYTYWWLDG; CWSSFAWYC.

It was shown that the binding of phages displaying those peptides could be competed off by addition of 10 μM of LF_{N}. This suggests that these peptides are allowing the phages to bind on a site close to, or structurally related to, the binding site of LF_{N}. This assumption was further supported by the fact that some of these peptides (asterisks-marked), when chemically synthesized and purified, could prevent radiolabeled LF_{N} from binding on the PA₆₃ heptamer.

Seven out of these eight peptides display the YWWL motif. This suggests that this motif might be critical in allowing binding, although this sequence can not be found in the “natural” ligands of the PA₆₃ heptamer (LF_{N}, EF_{N} or PA₂₀). However, a peptide with the sequence HTSTYWWLD could not compete with LF_{N} for binding on the heptamer, suggesting that the amino acids coming immediately after this motif also play a fundamental role in binding. No specific amino acid is conserved among the C-terminal residues of the peptides, which might suggest that only a backbone carboxyl is needed to allow binding. It might also be noted that a glycine is always found in this C-terminal part, although at different positions after the YWWL motif.

The peptide not showing the YWWL motif was isolated from a library of phages displaying “cysteine-constrained” peptides. Peptides displayed in this library are bordered by two cysteines, which can presumably form a disulfide bond. We have no indication that the disulfide bond is present nor necessary for binding of the isolated phage. The hydrophobicity of the sequence isolated is consistent with the hydrophobicity of the YWWL motif. The peculiar conformation of the “cysteine-constrained” peptide might explain why the motif was not isolated again. This strengthens the assumption that the hydrophobic residues are adopting a specific conformation upon binding, which is needed for binding.

The following references are incorporated herein, in their entirety.

1. Mammen, M., Choi, S.-K. & Whitesides, G. Polyvalent Interactions in Biological Systems: Implications for Design and Use of Multivalent Ligands and Inhibitors. Angew. Chem. Int. Ed. Engl., 37, 2754-2794 (1998).

2. Spaltenstein, A. & Whitesides, G. Polyacrylamides bearing Pendant-Sialoside Groups Strongly Inhibit Agglutination of Erythrocytes by Influenza Virus. J. Am. Chem. Soc. 113, 686-687 (1991).

3. Rao, J., Lahiri, J., Isaacs, L., Weiss, R. M. & Whitesides, G. M. A trivalent system from vancomycin.D-ala-D-Ala with higher affinity than avidin.biotin. Science 280, 708-711(1998).

4. Matrosovitch, M. N., Mochalova, L. U., Marinina, V. P., Byramova, N. E. & Bonvin, N. V. Synthetic polymeric sialoside inhibitors of influenza virus receptor-binding activity. FEBS Lett. 272, 209-212 (1990).

5. Gordon, E. J., Sanders, W. J. & Kiessling, L. L. Synthetic ligands point to cell surface strategies. Nature 392, 30-31 (1998).

6. Kramer, R. H. & Karpen, J. W. Spanning binding sites on allosteric proteins with polymer-linked ligand dimers. Nature 395, 710-713 (1998).

7. Kitov, P. I. et al. Shiga-like toxins are neutralized by tailored multivalent carbohydrate ligands. Nature 403, 669-672 (2000).

8. Dixon, T. C., Meselson, M., Guillemin, J. & Hanna, P. C. Anthrax. N. Engl. J. Med. 341, 815-826 (1999).

9. Leppla, S. H. Anthrax Toxin. Bacterial toxins and virulence factors in diseases, (Dekker, New York, 1995) pp. 543-572.

10. Petosa, C., Collier, R. J., Klimpel, K. R., Leppla, S. H. & Liddington R. C. Crystal structure of the anthrax toxin protective antigen. Nature 385, 833-838 (1997).

11. Duesbery, N. S. et al. Proteolytic inactivation of MAP-kinase-kinase by anthrax lethal factor. Science 280, 734-737 (1998).

12. Zwick, M. B., Shen, J. & Scott, J. K. Phage-displayed peptide libraries. Curr. Opin. Biotechnol. 9, 427-435 (1998).

13. Arora N. & Leppla S. H. Residues 1-254 of anthrax toxin lethal factor are sufficient to cause cellular uptake of fused polypeptides. J. Biol. Chem. 268, 3334-3341 (1993).

14. Milne, J. C., Blanke, S. R., Hanna, P. C. & Collier, R. J. Protective antigen-binding domain of anthrax lethal factor mediates translocation of a heterologous protein fused to its amino- or carboxy-terminus. Mol. Microbiol., 15, 661-666 (1995).

15. Ezzell, J. W., Ivins, B. E. & Leppla, S. H. Immunoelectrophoretic analysis, toxicity, and kinetics of in vitro production of the protective antigen and lethal factor components of Bacillus anthracis toxin. Infect. Immun., 45, 761-767 (1984).

16. Milne, J. C., Furlong, D., Hanna, P. C., Wall, J. S. & Collier, R. J. Anthrax protective antigen forms oligomers during intoxication of mammalian cells. J. Biol. Chem., 269, 20607-20612 (1994).

17. Koivunen, E. et al. Tumor targeting with a selective gelatinase inhibitor. Nat. Biotechnol. 17, 768-774 (1999).

18. Wrighton, N. C. et al. Small Peptides as Potent Mimetics of the Protein Hormone Erythropoietin. Science 273, 458-463 (1996).

19. Miller, C. J., Elliott, J. L. & Collier, R. J. Anthrax protective antigen: prepore-to-pore conversion. Biochemistry 38, 10432-10441 (1999).

20. Benson, E. L., Huynh, P. D., Finkelstein, A. & Collier, R. J. Identification of residues lining the anthrax protective antigen channel. Biochemistry 37, 3941-3948 (1998).

21. Zhao, J., Milne, J. C. & Collier, R. J. Effect of anthrax toxin's lethal factor on ion channels formed by the protective antigen. J. Biol. Chem., 270, 18626-18630 (1995).

22. Mammen, M., Dahmann, G. & Whitesides G. Effective inhibitors of hemagglutination by influenza virus synthesized from polymers having active ester groups. Insight into mechanism of inhibition. J. Med. Chem. 38, 4179-4190 (1995).

23. Pace, C. N., Vajdos, F., Fee, L., Grimsley, G. & Gray, T. How to measure and predict the molar absorption coefficient of a protein. Protein Sci. 4, 2411-2423 (1995).

24. Wesche, J., Elliott, J. L., Falnes, P. O., Olsnes, S. & Collier, R. J. Characterization of membrane translocation by anthrax protective antigen. Biochemistry 37, 15737-15746 (1998).

What is claimed is:

1. A method for treating a mammal suffering from or susceptible to an infectious agent, comprising administering to the mammal an effective amount of a polymer having linked thereto a plurality of therapeutic agents, and wherein the polymer comprises polymerized dextran units or polymerized ethylene glycol units.

2. The method of claim 1 wherein one or more of the plurality of therapeutic agents is a peptide.

3. The method of claim 1 or 2 wherein one or more of the therapeutic agents are covalently linked to the polymer.

4. The method of any one of claims 1 through 3 wherein the mammal is suffering from a gram-negative bacterial infection.

5. The method of any one of claims 1 through 3 wherein the mammal is suffering from a gram-positive bacterial infection.

6. The method of any one of claims 1 through 3 wherein the mammal has an anthrax infection.

7. The method of any one of claims 1 through 3 wherein the mammal is suffering from an infection caused by infectious disease agents.

8. The method of any one of claims 1 through 3 wherein the infectious disease agent is a virus, fungi, parasite, or protozoa.

9. The method of any one of claims 1 through 8 wherein polymer comprises polymerized dextran units.

10. The method of any one of claims 1 through 8 wherein polymer comprises polymerized ethylene glycol units.

11. The method of any one of claims 1 through 10 wherein the polymer comprises at least about forty polymerized dextran units or polymerized ethylene glycol units.

12. The method of claim 11 wherein the polymer comprises at least about one hundred polymerized dextran units or polymerized ethylene glycol units.

13. The method of any one of claims 1 through 12 wherein one or more of the therapeutic agents inhibit the functioning of the heptameric complex of anthrax toxin.

14. The method of any one of claims 1 through 13 wherein the polymer comprises pendant hydrophobic moieties.

15. The method of any one of claims 1 through 13 wherein the polymer comprises pendant hydrophilic moieties.

16. The method of any one of claims 1 through 15 wherein the ratio of the peptides to polymerized dextran units or polymerized ethylene glycol units is at least about one peptide per ten dextran units or polymerized ethylene glycol units.

17. The method of any one of claims 1 through 15 wherein the ratio of the peptides to polymerized ethylene glycol units at least about one peptide per ten ethylene glycol units.

18. The method of any one of claims 1 through 17 wherein one or more of the therapeutic agents can inhibit the functioning of the heptameric complex of anthrax toxin.

19. The method of claim 18 wherein one or more of the therapeutic agents can interfere with the binding of edema factor and lethal factor of the anthrax toxin.

20. The method of any one of claims 1 through 19 wherein one or more of the therapeutic agents can interfere with the mechanism of action of infectious disease agent toxins.

21. The method of any one of claims 2 through 19 wherein one or more of the peptides has a total of from about 5 to about 12 amino acids.

22. The method of any one of claims 2 through 19 wherein one or more of the peptides has a total of about 20 amino acids.

23. The method of any one of claims 1 through 21 wherein the polymer is crosslinked to another polymer.

24. The method of any one of claims 1 through 23 wherein one or more of the therapeutic agents are selected from the group consisting of oligonucleotides, proteins, enzymes, nucleic acids, or polynucleotides.

25. A method for treating a mammal suffering from or susceptible to anthrax, comprising administering to the mammal an effective amount of a polymer having covalently linked a plurality of pharmaceutically active compounds, and wherein the polymer comprises polymerized dextran units or polymerized ethylene glycol units.

26. The method of claim 25 wherein one or more of the pharmaceutically active compounds is a peptide.

27. The method of claim 25 wherein one or more of the pharmaceutically active compounds are oligonucleotides, proteins, enzymes, nucleic acids, or polynucleotides.

28. The method of claims 25 through 27 wherein the polymer comprises dextran units.

29. The method of claims 25 through 27 wherein the polymer comprises polymerized ethylene glycol units.

30. A method for treating bacterially infected mammalian cells, comprising contacting the cells an effective amount of a polymer having linked thereto a plurality of agents against the disease, and wherein the polymer comprises polymerized dextran units or polymerized ethylene glycol units.

31. The method of claim 30 wherein one or more of the plurality of therapeutic agents is a peptide.

32. The method of claim 30 or 31 wherein one or more of the plurality of therapeutic agents are oligonucleotides, proteins, enzymes, nucleic acids, or polynucleotides.

33. The method of any one of claims 30 through 32 wherein one or more of the therapeutic agents are covalently linked to the polymer.

34. The method of any one of claims 30 through 33 wherein the cells are infected with a gram-negative bacteria.

35. The method of any one of claims 30 through 33 wherein the cells are infected with a gram-positive bacteria.

36. The method of any one of claims 30 through 33 wherein the cells are infected with anthrax.

37. The method of any one of claims 30 through 33 wherein the cells are infected with an infectious disease agent which includes viruses, fungi, protozoa, parasites.

38. The method of any one of claims 30 through 37 wherein the polymer comprises dextran units.

39. The method of any one of claims 30 through 38 wherein one or more of the pharmaceutically active compounds are oligonucleotides, proteins, enzymes, nucleic acids, or polynucleotides.

40. A pharmaceutical composition comprising a polymer having covalently linked thereto a plurality of pharmaceutically active compounds, and wherein the polymer comprises polymerized dextran units or polymerized ethylene glycol units.

41. The composition of claim 40 wherein one or more of the plurality of therapeutic agents is a peptide.

42. The composition of claim 40 wherein one or more of the plurality of therapeutic agent compounds are oligonucleotides, proteins, enzymes, nucleic acids, or polynucleotides.

43. The composition of any one of claims 40 through 42 wherein one or more of the pharmaceutically active compounds can interfere with the binding of edema factor and lethal factor of the anthrax toxin.

44. The composition of any one of claims 40 through 43 wherein one or more of the pharmaceutically active compounds can interfere with the mechanism of action of infectious disease agent toxins.

45. The composition of any one of claims 40 through 44 wherein one or more of the peptides has a total of from about 5 to 12 amino acids.

46. The composition of any one of claims 40 through 44 wherein one or more of the peptides has a total of about 20 amino acids.

47. The composition of any one of claims 40 through 44 wherein the polymer is cross-linked to another polymer.

48. The composition of any one of claims 40 through 47 wherein one or more of the pharmaceutically active compounds are oligonucleotides, proteins, enzymes, nucleic acids, or polynucleotides.
